(19) United States
(12) Patent Application Publication
(10) Pub. No.: US 2022/0015329 A1
(43) Pub. Date: Jan. 20, 2022

(54) METHOD FOR ACQUIRING ANIMAL NOSE PATTERN IMAGE

(71) Applicant: ISCILAB CORPORATION, Seoul (KR)

(72) Inventors: Hyeong In CHOI, Seoul (KR); Stephanie Sujin CHOI, Seoul (KR)

(21) Appl. No.: 17/268,188

(22) PCT Filed: Aug. 8, 2019
(86) PCT No.: PCT/KR2019/009964

§ 371 Date: Sep. 7, 2021

(30) Foreign Application Priority Data
Aug. 13, 2018 (KR) 10-2018-0094389

(51) Int. Cl. A01K 11/00; G06K 9/00; G06K 9/20

(52) U.S. Cl. CPC A01K 11/006; G01N 2021/7786; G06K 9/2036; G06K 9/00885

(57) ABSTRACT

The present invention is a method for overcoming the challenge of acquiring good quality animal nose pattern images for biometric recognition caused by the moisture present on the nose surface, by removing the moisture or by pairing moisture removal and the application of an astringent or pigment prior to image capture.

### FIELD OF THE INVENTION

The present invention addresses the challenge of acquiring good quality animal nose pattern images for biometric recognition using a camera caused by the moisture present on the nose surface, and provides as a solution the removal of the moisture or the removal of the moisture followed by the application of astringents or pigments to the nose surface.

### BACKGROUND OF THE INVENTION

Like the human fingerprint that is unique to each individual, certain animals—almost all species belonging to the suborders Ruminatidae, Canidae, and Felidae—are known to have unique nose patterns. Therefore, attempts have been made to develop an animal biometric recognition system using nose patterns. The first step for developing this system is to reduce the burden on the nose pattern matching algorithm by acquiring nose pattern images of the highest quality possible.

There are two different approaches for obtaining animal nose patterns: the contact method and non-contact method.

The contact method includes making ink impression prints as well as using capacitive sensors like those used for fingerprints. The ink impression method has long been used in Japan for the identification cattle. However, this method has a high risk of creating distorted impressions by applying too much ink that causes the loss of pattern detail, or uneven pressure. The prior art is as follows:

(Patent Document 1) Korean Patent Registration No. 10-1527801

(Patent Document 2) Korean Patent Registration No. 10-1494717

(Patent Document 3) Korean Patent Registration No. 10-1494716

### DISCLOSURE OF THE INVENTION

### Technical Problem

The objective of the present invention is to provide a method for removing the moisture on the surface of an animal's nose to minimize or prevent obstructive reflections, for the acquisition of good quality nose pattern images.

Another objective of the present invention is to provide a method for removing the moisture from the nose surface followed by applying an astringent prior to capture for the acquisition of good quality nose pattern images.

Another objective of the present invention is to provide a method for removing the moisture from the nose surface followed by applying a pigment prior to capture for the acquisition of good quality nose pattern images.

Another objective of the present invention is to provide a method for selectively using UV, IR, or visible light illumination for the acquisition of good quality nose pattern images.

### Technical Solution

The solution provided by the present invention involves using a camera connected to or embedded in a mobile device or computer, or a standalone camera device to capture animal nose pattern images; generating nose pattern templates from the captured nose pattern images, wherein the template generation occurs on the camera device, mobile device, computer, or cloud server; and removing the moisture from nose surface prior to image capture to minimize or prevent reflections.

Another solution provided by the present invention is a method for removing the moisture from the nose surface followed by applying an astringent prior to capture for the acquisition of good quality nose pattern images.

Another solution provided by the present invention is a method for removing the moisture from the nose surface followed by applying a pigment prior to capture for the acquisition of good quality nose pattern images.

Another solution provided by the present invention is a method for selectively using UV, IR, or visible light illumination for the acquisition of good quality nose pattern images.

### Advantageous Effects

The effect of the present invention is the advantage of obtaining good quality nose pattern images by removing the moisture on the surface of an animal's nose to minimize or prevent obstructive reflections.

Another effect of the present invention is the advantage of obtaining good quality nose pattern images by removing the moisture from the nose surface, further enhanced by also applying an astringent prior to capture.

Another effect of the present invention is the advantage of obtaining good quality nose pattern images by removing the moisture from the nose surface, further enhanced by also applying a pigment prior to capture.

Another effect of the present invention is the advantage of obtaining good quality nose pattern images, further enhanced by selectively using UV, IR, or visible light illumination for image capture.

### BRIEF DESCRIPTION OF DRAWINGS

FIG. 1 shows a conventionally used contact-type (a) ink impression method, and (b) capacitive method.

FIG. 2 shows (a) a photographic image of a nose pattern, and (b) an outline drawing of the nose pattern obtained from the same image.

FIG. 3 shows the anatomical structure of a dog's nose, which comprises a nasal cavity and an anatomical structure to keep the nose surface moist.

FIG. 4 is an image of another animal's (deer) nose with moisture on the surface.

FIG. 5 is a nose image captured prior to wiping off the moisture from the surface.

FIG. 6 is an image of the same nose as FIG. 5 captured after wiping off the moisture from the surface.

### DETAILED DESCRIPTION OF THE INVENTION

There is provided a method for acquiring animal nose pattern images including: capturing a nose pattern image using a camera connected to or embedded in a mobile device or a computer, generating a nose pattern template for biometric registration or authentication from the captured nose pattern image, wherein the nose pattern template generation occurs on at least one out of the group consisting of the mobile device, the computer, or a cloud server; and removing a moisture from a surface of an animal's nose prior to the nose pattern image capture to minimize or prevent reflections.

There are three stages in the process of animal nose image acquisition in which the quality of the image can be dramatically improved: pre-capture, during capture, and post-capture.

During capture, the key factors are the luminance range of the subject and the dynamic range of the camera medium. When photographed without proper care, these translate into nose images with bright, obstructive reflections (extreme highlights) and shaded portions wherein the pattern details are obscured.

While the presence of some reflections in and of itself does not make a nose pattern image entirely unusable for biometric identification, it is a problem when the reflections encompass a large portion of the nose.

The amount of liquid substance present on a nose surface greatly affects the clarity of nose pattern images when photographically capturing nose images with a camera. In the present invention, the ‘liquid substance’ present on a nose surface is expressed as ‘wetness’ or ‘moisture’ depending on the context, but they all have the same meaning.

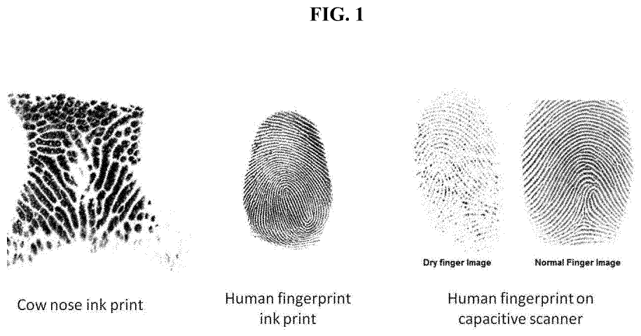

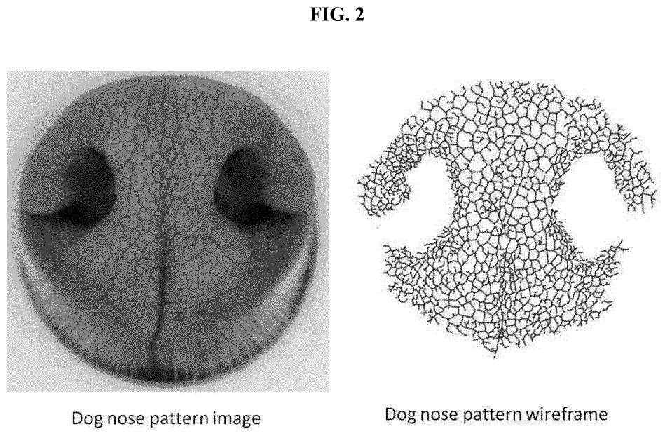

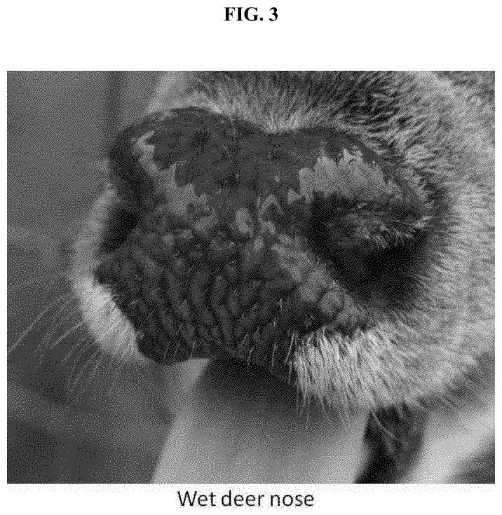

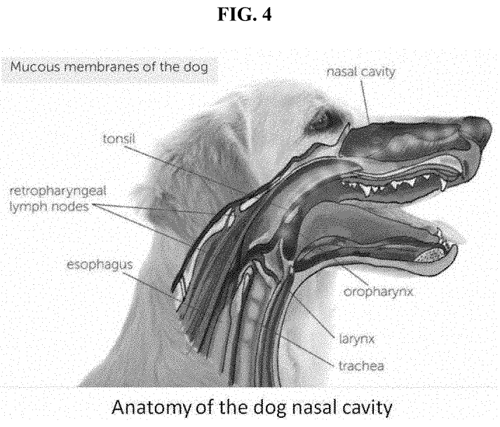

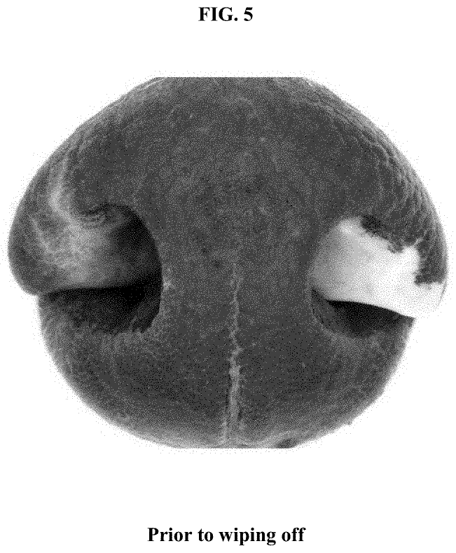

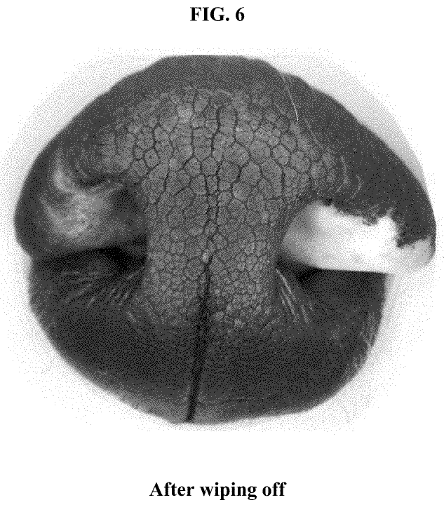

To briefly describe the drawings, FIG. 1 shows a conventionally used contact-type (a) ink impression method, and (b) capacitive method. FIG. 2 shows (a) a photographic image of a nose pattern, and (b) an outline drawing of the nose pattern obtained from the same image. FIG. 3 shows the anatomical structure of a dog's nose, which comprises a nasal cavity and an anatomical structure to keep the nose surface moist. FIG. 4 is an image of another animal's (deer) nose with moisture on the surface. FIG. 5 is a nose image captured prior to wiping off the moisture from the surface. FIG. 6 is an image of the same nose as FIG. 5 captured after wiping off the moisture from the surface.

The bright, white spots, by definition, white out the groove and bead detail thus reducing the size of usable real estate with valuable pattern data.

On the other hand, it is also possible to lose pattern details due to shadows. When the luminance range of the subject does not match the dynamic range of the camera medium, the areas in the shade—most likely as a result of the curvature of the nose—appear too dark with not enough contrast to discern a bead pattern.

Post-capture treatment involves the enhancing and processing of the images using an image editing software or algorithm to make them most suitable for pattern recognition.

However, the most crucial may in fact be the pre-capture stage, where the nose surface is prepared and primed to be in the ideal condition for photographic capture. The present invention explores such ways in which to ensure a higher quality output even before aiming the camera.

In this regard, a more recent non-contact approach is to use a camera to take pictures of a nose at a fairly close distance. However, this method presents the challenge of acquiring high-quality nose pattern images required for biometric recognition due to unpredictable and often unfavorable lighting conditions and a number issues mainly attributed to the intrinsic curvature of an animal's nose, the color of the nose, as well as the moisture on the nose surface.

In essence, a ‘good’ nose pattern image has sufficient contrast between the grooves and the beads, which correlates with the color of the nose and the illumination on the nose surface.

In the following description, numerous specific details are set forth. However, it is to be understood that any reasonable variations of the embodiments of the invention that retain the core concept should be considered within the scope of the present disclosure.

The meaning of specific terms of words used in the following sections should not be limited to the literal or commonly employed sense, but should be interpreted within the appropriate context.

Therefore “a” or “an” should be read to include one or at least one, and the singular word form of the element or component also includes the plural unless the number is obviously meant to be singular.

The implementation of any biometric identification technology begins with the acquisition of the particular biometric data.

At the core, the present invention relates to the acquisition of good quality animal nose pattern images for biometric registration and/or authentication, from which nose pattern templates are generated.

‘Nose pattern templates’ in the present invention refers to the nose pattern information format stored in the nose pattern database for registration and/or authentication. The nose pattern template is a nose pattern image format stored in the memory for use when computing similarity between nose pattern images. Therefore, the nose pattern template contains all of the relevant characteristic information for what makes each nose pattern unique, but its size is relatively small compared to the original nose pattern image captured with a camera.

For example, if the original image is about 100 Kbyte, then the corresponding nose pattern template would be about 10 Kbyte or about one-tenth of the original or less. This means that the nose pattern template will take up significantly less space in the memory than the original nose pattern image.

The nose pattern template, which contains the characteristic information that makes each nose pattern unique, is generated using such methods as the Fourier transform and the wavelet transform to increase processing speed during biometric recognition and reduce burden on the storage.

In this case it is the animal nose pattern image, for which there are basically two different approaches: contact type and non-contact type.

The contact method, the result of which is shown in FIG. 1 obtains nose images either by the application of a pigment onto the nose for a contact impression print, or by using a capacitive touch sensor as used for fingerprints. The ink impression method has been used for a long time in places like Japan to identify cows, yet not only is it a rather cumbersome and delicate process that requires skill, but also impractical and inaccurate for an animal with a small nose. Moreover, the contact method runs the risk of creating distorted impressions from applying too much or uneven pressure onto the nose.

Essentially, contact impression is a primitive method of mapping a 3D object onto a 2D surface, i.e. UV unwrapping. The contrast between the grooves and the beads of the nose pattern illustrated by the absence or presence of ink transfer or capacitive conduction by the three-dimensional physical contour and protrusions of the nose.

The non-contact method, the result of which is shown in FIG. 2 on the other hand, is digital UV unwrapping, which means the grooving and beading are denoted by the visual cues in terms of color and shade. In other words, the three-dimensional contours and protrusions of the nose are inferred from the two-dimensional, photographic elements of the captured image. Therefore, the visual quality of the nose pattern surface matters, and the present invention relates to all the ways the animal nose can be primed to enhance and accentuate the appearance of the surface features.

The most distinctive and problematic aspect of the animal nose is the wetness, as shown in FIGS. 3 and 4. There are a few different reasons an animal's nose is wet: usually there is a thin layer of mucus, irrigated throughout the nose crevices by way of the philtrum, that allows them to absorb scent chemicals; animals tend to lick their nose frequently to clean it and to lick off the collected scent chemicals so that the olfactory glands on the roof of their mouth can sample them; and some animals like dogs sweat through the nose to regulate body temperature.

First and at the very least, the nose needs to be properly and thoroughly blotted and wiped to remove the excess moisture and any dirt or foreign substance from the surface—both on top of and in between the beads. This can be done using an enormously wide range of materials and products; such as, any type of tissue, paper, or towel made with a variety of different fibers; any other cotton products like cotton balls and pads; and any type of sponge.

However, through many trials it was found that the simplest and most effective way to clean the nose in order to obtain clear nose pattern images as in FIG. 6 is by using a wet wipe—also known as a moist towelette, and is a small moistened piece of paper or cloth.

While dry materials were somewhat effective, the general idea seems to have been to get rid of the excess moisture while not desiccating the nose to the point of discomfort; typically, wiping using a dry towel resulted in an almost immediate licking response, which rendered the initial wiping moot. In any case there should be as little time between the wiping and the image capture as possible, before the nose has a chance to be rehydrated by various means.

Moreover, the wet wipe may also be infused with some oil, moisturizing agent, or anything else that could help with alleviate uncomfortable dryness on the nose, so long as the substance is safe to ingest.

The next possible step in the priming process is the application of an astringent onto the nose surface using an application tool, such as a sponge, brush, cloth, cotton ball or cotton pad. An astringent substance is a chemical compound that tends to constrict body tissues. Antiperspirants often contain astringents, like aluminum salts, that shrink the pores thereby reducing the amount of sweat secretion. Other options for astringents include a low concentration of hydrogen peroxide or herbal substances like witch hazel.

The aforementioned two steps are subtractive, where the intention is to remove and further temporarily prevent the wetness from the animal nose surface. On the other hand, the last step is additive, wherein some type of pigment is to be applied onto the beads in order to accentuate the visual contrast between the beads and the grooves.

In the most elementary implementation of this last step, any type of pigment can be used to thinly coat the outermost surface of the nose after removing the moisture as in FIG. 6, with care taken to not allow any pigment to seep into the grooves. Presumably, dark-colored pigments would be used on light-colored noses and light-colored pigments would be used on dark-colored noses, so as to maximize the color contrast between the painted bead tops and the unpainted grooves. Immediately following the pigment application, the nose pattern image may be captured using visible light illumination and visible light photography.

Many variations of this step are possible, the first of which involves to use of infrared (IR) reflective pigments like those containing titanium dioxide or aluminum powder. IR reflective pigments do not absorb in the near infrared range (NIR) whereas water absorbs NIR, which means under external NIR lighting the beads would reflect the light and appear white, while the grooves—especially if moisture has already returned—would appear dark or black. A nose pattern image captured using IR photography under such conditions would show highly identifiable patterns.

Similarly, ultraviolet (UV) reflective pigments like those containing aluminum powder or zinc oxide can be used to yield a similar result. However, the external lighting would have to be UV light, paired with a UV capable camera.

Another category of specialty pigments is the fluorescents. IR fluorescent pigments like Egyptian blue (made with artificial copper calcium silicate, i.e. CaCuSi₄O₁₀), Cadmium red (cadmium sulfoselenide, i.e. Cd(S,Se)), and Cadmium yellow (cadmium sulfide, i.e. CdS) fluoresce under visible light, and appear luminescent in IR photography.

Lastly, there is a wide variety of readily accessible every substances that are UV fluorescent, including quinine, which can be found in tonic water; vitamin A and the B vitamins thiamine, niacin, and riboflavin; petroleum jelly, like Vaseline; olive and canola oil; and honey. UV fluorescent agents fluoresce under UV light, and appear luminescent in visible photography.

In summary, the priming of the animal nose surface for nose pattern image acquisition comprises the removal of moisture from the nose, followed by one or more of the steps including an application of an astringent and application of a pigment.

The subsequent capturing of the nose pattern image is done using a standalone camera device, such as a point-and-shoot digital camera or DSLR camera; an external camera, such as a webcam, or a specially designed hand-held camera device, connected to a mobile device or computer wirelessly or via a wire cable; or a camera embedded in a mobile device or computer.

In order to improve the clarity of the nose pattern image prior to capture using various types of camera devices, if necessary it is possible to include the step of adjusting the contrast, brightness, saturation, and/or exposure levels in the camera.

Upon the acquisition of the nose pattern image, it is processed, if necessary, to improve the visibility of the patterns; wherein the processing step comprises image enhancing techniques as adjusting the contrast, brightness, saturation, and exposure levels.

These steps of nose pattern image acquisition, nose pattern template generation, and nose pattern biometric registration and/or authentication may be performed on various devices—including a mobile device, a camera, or a computer connected to a camera—that has the appropriate control program or application onboard.

Then, the processed nose pattern image is used to generate a nose pattern code; wherein the nose pattern code refers to any format of an image or data representation that is compatible for a particular biometric pattern recognition matching algorithm. For example, the nose pattern code may denote a collection of polygons that represent the shape and relative positions of the beads in a nose pattern; or a simplified line trace drawing of the nose pattern groove mesh.

The processing and generating steps can occur either on the standalone camera device, mobile device, computer, or on a cloud server via an Internet connection, depending on the specific configuration of the image acquisition system.

Finally, the generated nose pattern code is stored onto a storage unit; wherein the storage unit is located on the standalone camera device, mobile device, computer, or on the cloud server. The nose pattern code is stored temporarily to be matched against other codes already existing in a database of registered animals; or, if no matching record exists, stored permanently to be enrolled in the database.

For clarity, the following summarizes the method for acquiring animal nose pattern images as previously described.

The method for acquiring nose pattern images comprises: capturing nose pattern images using a mobile device, a camera connected to or embedded in a computer, or a standalone camera; and generating nose pattern templates from the captured nose pattern images to use for biometric registration and/or authentication.

The above nose pattern image capture and template generation steps may be performed on a mobile device, a standalone camera device, a computer and/or a cloud server; wherein moisture is removed from the nose surface prior to image capture in order to minimize or prevent reflections.

After removing moisture from the nose surface, an astringent and/or a pigment may also be applied to the nose surface to obtain clear nose pattern images.

Moreover, UV, IR, or visible light illumination may also be used to obtain clear nose pattern images.

The pigment used may be selected from at least one of a UV reflective pigment, a UV fluorescent pigment, an IR reflective pigment, and an IR fluorescent pigment.

The UV reflective pigment may be selected from at least one of aluminum powder and zinc oxide.

The UV fluorescent pigment may be selected from at least one of quinine, vitamin A, vitamin B, petroleum jelly, olive oil, canola oil, and honey.

The IR reflective pigment may be selected from at least one of titanium dioxide and aluminum powder.

The IR fluorescent pigment may be selected from at least one of Egyptian blue (made with artificial copper calcium silicate), Cadmium red (cadmium sulfoselenide), and Cadmium yellow (cadmium sulfide).

Moisture on the nose surface may be removed using a sponge, a wet wipe, paper, or a tissue or fabric made of a variety of different fibers including microfiber, synthetic fibers, and cotton; and these may be dry, moist, or be infused with oils or antibacterial agents.

The preferred method of removing moisture is using a wet wipe.

The astringent may be selected from one of an antiperspirant, hydrogen peroxide, aluminum salts, and witch hazel.

UV fluorescent pigments may be applied onto the nose surface during nose pattern image capture using UV light illumination.

IR fluorescent pigments may be applied onto the nose surface during nose pattern image capture using visible light illumination.

In order to obtain good quality nose pattern images, at least one of contrast, brightness, saturation, and/or exposure level in the camera may be adjusted prior to capture.

The acquired nose pattern images may also be adjusted for at least one of contrast, brightness, or saturation before generating nose pattern templates biometric registration and/or authentication.

It is also possible to specify various protection ranges based on the above-described matters.

### INDUSTRIAL APPLICATIONS

The present invention identifies the key challenge in capturing good quality animal nose pattern images using a camera as being the persistent presence of a layer of moisture on the nose surface. The solution presented involves removing the moisture and also optionally applying astringents or pigments prior to image capture to enable the acquisition of good quality nose pattern images, from which nose pattern templates may be generated for biometric registration and/or authentication. The described method is highly applicable for biometric identification technology in the animal industry.

1. A method for acquiring animal nose pattern images, comprising:
capturing a nose pattern image using a camera connected to or embedded in a mobile device or a computer,
generating a nose pattern template for biometric registration or authentication from the captured nose pattern image,
wherein the nose pattern template generation occurs on at least one out of the group consisting of the mobile device, the computer, or a cloud server; and
removing a moisture from a surface of an animal's nose prior to the nose pattern image capture to minimize or prevent reflections.

2. The method of claim 1, further comprising applying an astringent to the nose surface prior to the nose pattern image capture.

3. The method of claim 1, further comprising applying a pigment to the nose surface prior to the nose pattern image capture.

4. The method of claim 1, further comprising using illumination by an external light source during the nose pattern image capture, wherein the external light source is at least one out of a group consisting of UV light, IR light, and visible light.

5. The method of claim 3, wherein the pigment is at least one out of a group consisting of a UV reflective pigment, a UV fluorescent pigment, an IR reflective pigment, or and IR fluorescent pigment.

6. The method of claim 5, wherein the UV reflective pigment is at least one out of a group consisting of aluminum powder and zinc oxide.

7. The method of claim 5, wherein the UV fluorescent pigment is at least one out of a group consisting of quinine, vitamin A, vitamin B, petroleum jelly, olive oil, canola oil, and honey.

8. The method of claim 5, wherein the IR reflective pigment is at least one out of a group consisting of titanium dioxide and zinc oxide.

9. The method of claim 5, wherein the UV fluorescent pigment is at least one out of a group consisting of artificial copper calcium silicate, cadmium sulfoselenide, and cadmium sulfide.

10. The method of claim 1,
wherein the moisture is removed using at least one material out of a group consisting of a sponge, a wet wipe, a paper, a fabric, microfiber, synthetic fibers, and cotton; and
wherein the above material is at least one out of a group consisting of dry, moist, oil-infused, or antibacterial-infused.

11. The method of claim 1, wherein the moisture is removed using a wet wipe.

12. The method of claim 2, wherein the astringent is at least one out of a group consisting of an antiperspirant, hydrogen peroxide, aluminum chloride, and witch hazel.

13. The method of claim 5, further comprising using the UV fluorescent pigment and an illumination by an external UV light source.

14. The method of claim 5, further comprising using the IR fluorescent pigment and an illumination by an external visible light source.

15. The method of claim 1, further comprising adjusting at least one out of a group consisting of a contrast, a brightness, a saturation, and an exposure level in the camera's settings prior to nose pattern image capture.

16. The method of claim 1, further comprising adjusting at least one out of a group consisting of a contrast, a brightness, and a saturation level of the captured nose pattern image prior to the nose pattern template generation.
